(19) United States
(12) Patent Application Publication
(10) Pub. No.: US 2022/0265308 A1
(43) Pub. Date: Aug. 25, 2022

(54) TREATMENT DEVICE WITH DAMPING FEATURE

(71) Applicant: OLYMPUS MEDICAL SYSTEMS CORP., Tokyo (JP)

(72) Inventors: Hiroshi ASHIBA, Tokorozawa-shi (JP); Yasuhiro MAEDA, Tokyo (JP); Minoru KATSUMATA, Tokyo (JP)

(73) Assignee: OLYMPUS MEDICAL SYSTEMS CORP., Tokyo (JP)

(21) Appl. No.: 17/667,587

(22) Filed: Feb. 9, 2022

Related U.S. Application Data
(60) Provisional application No. 63/152,884, filed on Feb. 24, 2021.

(51) Int. Cl. A61B 17/32; A61B 18/14

(52) U.S. Cl. CPC A61B 17/320092; A61B 18/1445; A61B 2018/00601

(57) ABSTRACT

Treatment device for ultrasonic treatment and high frequency treatment procedure is equipped with an ultrasonic transducer including piezoelectric elements converting electrical power into ultrasonic vibrations. The treatment device includes a transmission rod with a treatment probe and jaw for clasping objects. The transmission rod includes features for damping, such as a sheath, a coating, a geometry of the outer surface of the transmission rod, or combinations of such features, to minimize or prevent excess vibrations and to, among other things, decrease frictional heat caused by the friction between the damping features and the transmission rod arising from attenuating the ultrasonic vibrations.

### RELATED APPLICATION DATA

This application is based on and claims priority under 35 U.S.C. § 119 to U.S. Provisional Application No. 63/152,884 filed on Feb. 24, 2021, the entire contents of which are incorporated herein by reference.

### FIELD OF DISCLOSURE

The present invention relates to an ultrasonic treatment device used for dissecting and coagulating tissues. The ultrasonic treatment device is equipped with ultrasonic transducer including piezoelectric elements converting electrical power into ultrasonic vibrations. The ultrasonic vibrations are transmitted along the transmission member to a probe that serves to clasp objects together with a jaw. The transmission member may create undesired transverse vibration that causes problems such as deterioration of blood vessel sealing performance, heat generation, abnormal stress, and abnormal noise.

### BACKGROUND

In the discussion that follows, reference is made to certain structures and/or methods. However, the following references should not be construed as an admission that these structures and/or methods constitute prior art. Applicant expressly reserves the right to demonstrate that such structures and/or methods do not qualify as prior art against the present invention.

FIG. 10 is a figure of an ultrasonic treatment device in the related art (U.S. Pat. No. 8,696,666). The related art surgical operation system 1 consists of a handpiece 2, a main body apparatus 3 which is an output control apparatus, a foot switch 4 and a counter electrode plate 5. The handpiece 2 is a surgical treatment instrument capable of treatment using both ultrasonic and high-frequency current. The handpiece 2 is connected to the main body apparatus 3 via a cable 2 a which is attachable and detachable. The handpiece 2 has an insertion portion 2b and a handle portion 2c. The connector portion 3a connects the handpiece to the main body apparatus 3, which controls the output of the ultrasonic vibration and/or high-frequency current. The main body apparatus 3 has a plurality of displays 3b and a plurality of various operation buttons 3c for controlling the performance of handpiece 2. The foot switch 4 is connected to the main body apparatus 3 through a cable 4 a, and switches the mode from treatment using ultrasonic vibration, treatment using high-frequency current, or treatment using both. The counter electrode plate 5 is connected to the main body apparatus 3 through a cable 5 a. The counter electrode plate 5 is a return electrode for returning a current which passes through a subject at the time of monopolar output of a high-frequency current.

FIG. 11 is a figure of a portion of an ultrasonic treatment device in the related art (U.S. Pat. No. 5,989,275). The related art ultrasonic treatment device includes a transmission rod 86 used for transmitting ultrasonic vibrations to the ultrasonic probe. The transmission rod 86 is covered by a damping sheath 160, which is further covered by the elongated tubular member 174. Diametrically opposed openings 162b and 162c, as well as longitudinal slit 164 are formed on the damping sheath 160. Compliant members 190b and 190c (O-rings and fenders) are disposed around the periphery of the damping sheath 160, which are preferably disposed around the nodes to minimize damping of the desired longitudinal vibration.

The damping sheath 160 is constructed of a polymeric material, preferably with a low coefficient of friction to minimize dissipation of energy from the axial motion or longitudinal vibration of the transmission rod 86. The damping sheath 160 is preferably in light contact with the transmission rod 86 to dampen or limit non-axial or transverse side-to-side vibration of the transmission rod 86. The damping sheath 160 can dampen transverse motion occurring near multiple nodes and antinodes of the unwanted vibration which are located randomly along the length of the transmission rod 86 relative to the nodes and antinodes of the desired longitudinal vibration.

Transverse vibrations occurring in ultrasonic treatment devices when the ultrasonic probe is vibrated can lead to problems, such as deterioration of blood vessel sealing performance, heat generation, abnormal stress, and abnormal noise. Even though previous ultrasonic treatment devices may have structures, such as the damping sheath 160, such a damping sheath 160 is in contact throughout the transmission rod 86 in areas where dampening or limiting the non-axial or transverse side-to-side vibration is not necessary. Additionally, this configuration may cause problems such as heat generation through friction between the transmission rod 86 and the damping sheath 160 due to longitudinal vibration.

### SUMMARY

Accordingly, there is a need for designing an ultrasonic treatment device with an efficient structure in view of the practical usage, which would substantially obviate one or more of the issues due to limitations and disadvantages of related art treatment devices. An object of the present disclosure is to provide an improved treatment device having an efficient structure and practical administration of the associated medical procedure. For example, there is a need to provide improved damping solutions that, for example, minimize the contact between a transmission rod and a damping structure, such as a sheath, so as to minimize or prevent heat generation or other issues to arise. At least one or some of the objectives is achieved by the treatment device disclosed herein.

Additional features and advantages will be set forth in the description that follows, and in part will be apparent from the description, or may be learned by practice of the invention. The objectives and other advantages of the disclosed treatment device will be realized and attained by the structure particularly pointed out in the written description and claims thereof, as well as the appended drawings.

In general, the disclosed structures and systems provide for an ultrasonic treatment device efficiently suppressing problems such as deterioration of blood vessel sealing performance, heat generation, abnormal stress, and abnormal noise created from vertical and/or horizontal ultrasonic vibrations.

Embodiments of the disclosed surgical treatment device comprises a transducer generating ultrasonic vibrations, a transmission rod including a treatment probe in which a proximal end of the transmission rod is operatively connected to the transducer for transmitting ultrasonic vibration generated by the transducer to the treatment probe located at the distal end, and a damping feature for attenuating vibrations. The damping feature has an interior surface that circumscribes a first region of the transmission rod, and the interior surface of the damping feature is in contact with a first portion of the outer surface of the first region of the transmission rod and the interior surface of the damping feature is in non-contact with a second portion of the outer surface of the first region of the transmission rod. Furthermore, the first region includes at least one antinode of the transverse vibration.

In some embodiments, the treatment probe includes a curved portion.

In some embodiments, the first region includes a notch.

In some embodiments, the first portion comprises two opposing outer surfaces of the first region.

In some embodiments, the two opposing outer surfaces include the horizontal plane parallel to the direction of the curve of the curved portion.

In some embodiments, the second portion comprises two opposing outer surfaces of the first region that are flat and parallel to each other.

In some embodiments, the first portion does not include an antinode of the longitudinal vibration.

In some embodiments, the damping feature is a sleeve.

In some embodiments, the damping feature is a tube.

In some embodiments, the damping feature is a coating material.

In some embodiments, damping feature includes a slit.

In some embodiments, the transmission rod is configured as an electrode for treatment using high frequency currents.

In some embodiments, the surgical treatment device comprises a transducer generating ultrasonic vibration, a transmission rod including a treatment probe in which a proximal end of the transmission rod is operatively connected to the transducer for transmitting ultrasonic vibration generated by the transducer to the treatment probe located at the distal end, and a damping feature for attenuating vibrations. The damping feature has an interior surface that circumscribes an outer surface of a first region of the transmission rod, and the interior surface of the damping feature is in contact with a first portion of the circumferential surface of the first region of the transmission rod and the interior surface of the damping feature is in non-contact with a second portion of the circumferential surface of the first region of the transmission rod. Furthermore, the first region includes at least one antinode of the transverse vibration.

In some embodiments, the treatment probe includes a curved portion.

In some embodiments, the first portion includes a node of a longitudinal vibration.

In some embodiments, the first portion does not include an antinode of a longitudinal vibration.

In some embodiments, the first portion does not include a node of the transverse vibration.

In some embodiments, the damping feature is a sleeve.

In some embodiments, the damping feature is a tube.

In some embodiments, the damping feature is a coating material.

In some embodiments, the damping feature includes a slit.

In some embodiments, the transmission rod is configured as an electrode for treatment using high frequency currents.

In some embodiments, a transmission rod comprises an elongate body configured for transmitting ultrasonic vibration from a proximal end to a distal end and a treatment probe formed at the distal end of the elongate body, wherein the treatment probe includes a treatment surface and a curved portion. The elongate body includes a notch covering the vertical vertex of the elongate body and the notch includes an antinode of the transverse vibration.

In some embodiments, the notch does not include a node of the transverse vibration.

In some embodiments, the notch does not include an antinode of the longitudinal vibration.

In some embodiments, the notch includes a node of the longitudinal vibration.

In some embodiments, the transmission rod is configured as an electrode for treatment using high frequency currents.

In some embodiments, a transmission rod comprises an elongate body configured for transmitting ultrasonic vibration from a proximal end to a distal end and a treatment probe formed at the distal end of the elongate body, wherein the treatment probe includes a treatment surface and a curved portion. The elongate body includes a first region having a first circumferential outer surface and a second region having a second circumferential outer surface, where a diameter of the first circumferential outer surface is larger than a diameter of the second circumferential outer surface and the first circumferential outer surface includes an antinode of the transverse vibration.

In some embodiments, the first circumferential outer surface does not include a node of the transverse vibration.

In some embodiments, the first circumferential outer surface includes a node of the longitudinal vibration.

In some embodiments, the first circumferential outer surface does not include an antinode of the longitudinal vibration.

In some embodiments, the transmission rod is configured as an electrode for treatment using high frequency currents.

Other systems, methods, features and advantages will be, or will become, apparent to one with skill in the art upon examination of the following figures and detailed description. It is intended that all such additional systems, methods, features and advantages be included within this description, be within the scope of the present disclosure, and be protected by the following claims. Nothing in this section should be taken as a limitation on those claims. Further aspects and advantages are discussed below in conjunction with the embodiments of the disclosed input device. It is to be understood that both the foregoing general description and the following detailed description of the disclosed input device are examples and explanatory and are intended to provide further explanation of the disclosed input device as claimed.

### BRIEF DESCRIPTION OF THE DRAWING

The following detailed description of preferred embodiments can be read in connection with the accompanying drawings in which like numerals designate like elements and in which:

FIG. 1 shows an embodiment of a treatment device.

FIG. 2 shows a magnified view of the treatment end of the treatment device in Area P in FIG. 1.

FIG. 3A is a top view of a treatment region of an ultrasonic probe and FIG. 3B is an exaggerated representation, based on a simulation, of the ultrasonic vibrations of the treatment region in transverse vibration mode.

FIG. 4A is a side view of a treatment region of an ultrasonic probe and FIG. 4B is an exaggerated representation, based on a simulation, of the ultrasonic vibrations of the treatment region in transverse vibration mode.

FIG. 5 is an exaggerated perspective view of a treatment region of an ultrasonic probe and showing the variation in transverse vibration during vibration of the ultrasonic probe.

FIGS. 6A to 6C illustrates a damping structure and associated features of the transmission member of an ultrasonic probe transverse vibration.

FIGS. 7A to 7C illustrates a damping structure and associated features of the transmission member of an ultrasonic probe transverse vibration

FIG. 8A to 8D illustrates an alternative damping structure to that in FIGS. 6 and 7.

FIG. 9A to 9C illustrates an alternative damping structure to that in FIGS. 6, 7, and 8 including a tapered structure.

FIG. 10 shows an ultrasonic treatment device in the related art.

FIG. 11 shows a portion of an ultrasonic treatment device in the related art.

Throughout all of the drawings, dimensions of respective constituent elements are appropriately adjusted for clarity. For ease of viewing, in some instances only some of the named features in the figures are labeled with reference numerals.

### DETAILED DESCRIPTION

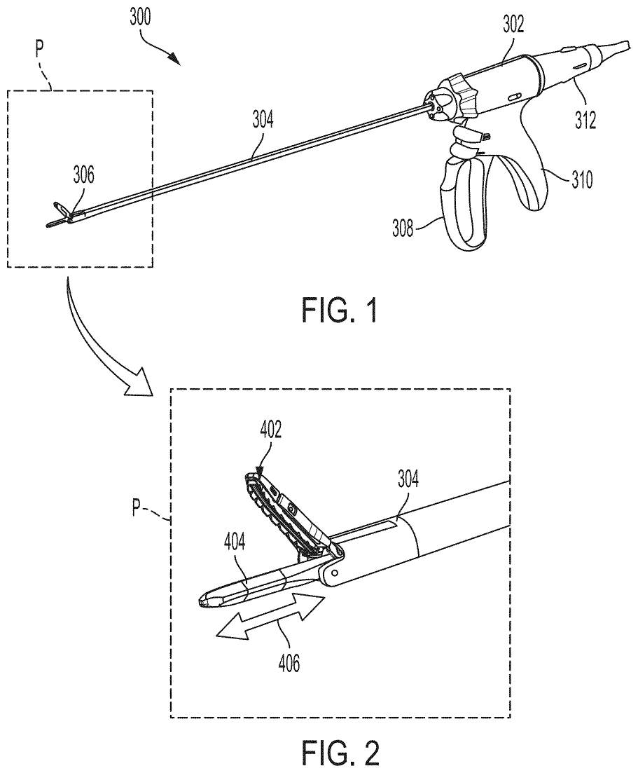

FIG. 1 is an illustration of a surgical treatment device 300 including a body 302, a shaft 304, and a treatment end 306. The body 302 includes a moving arm 308, a grip 310, and a transducer 312. The moving arm 308 is used together with grip 310 to actuate and operate the functions of treatment end 306. The transducer 312 includes an ultrasonic transducer which is connected to a power source supplying power used for ultrasonic treatment, as well as high-frequency treatment of surgical treatment device 300. The power source can be a wired or wireless power source. The shaft 304 protects the wires and members within, necessary for operating the functions of treatment end 306.

FIG. 2 is the magnified illustration of the treatment end 306 of the surgical treatment device 300. The treatment end 306 consists of a jaw 402 and an ultrasonic probe 404. In the current embodiment, the jaw 402 and the ultrasonic probe 404 open and close in the vertical direction through the manipulation of the movable handle 308 in order to grasp tissues and other objects for treatment, but ultrasonic probe 404 may be used for the treatment procedures without a jaw. The ultrasonic probe 404 vibrates at an ultrasonic frequency transmitted through the transmission member within shaft 304. A longitudinal vibration, an ultrasonic vibration of the ultrasonic probe 404 made in the direction 406, creates frictional heat used for treatment purposes such as dissection of tissues, as well as frictional heat caused through contacting objects such as damping members. The ultrasonic probe 404 may also serve as an electrode for treatment using high frequency currents.

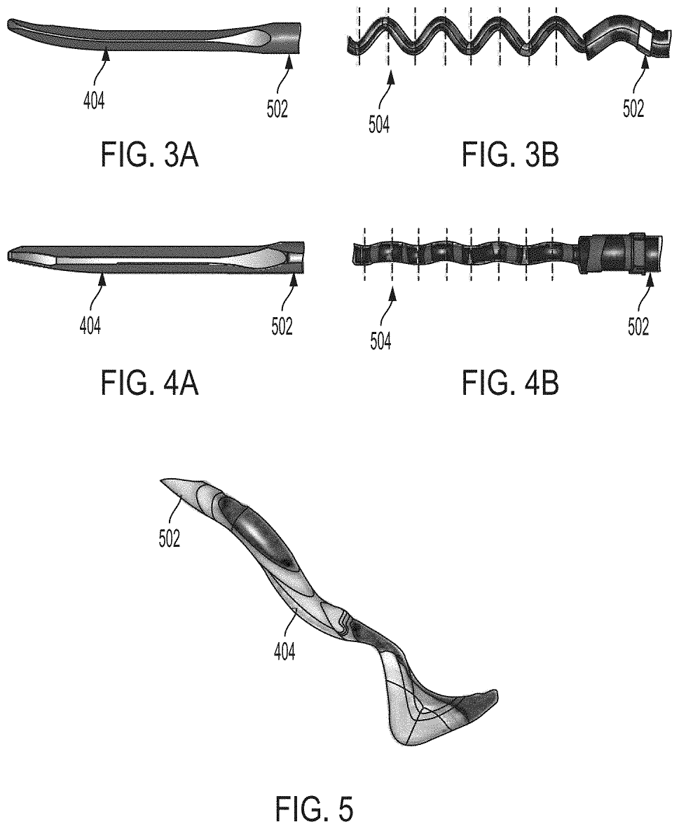

FIG. 3A illustrates the ultrasonic probe 404 viewed from the vertical direction, the direction the jaw 402 opens and closes. FIG. 3A also illustrates the transmission member 502 extending from the ultrasonic probe 404, extending within the shaft 304, and connecting to the transducer 312. The ultrasonic probe 404 and transmission member 502 are in its stationary state, a state where neither the ultrasonic vibration nor the high frequency current is applied to the ultrasonic probe 404 and transmission member 502.

FIG. 3B also illustrates the ultrasonic probe 404 viewed from the vertical direction, the direction the jaw 402 opens and closes. FIG. 3B illustrates an exaggerated representation of the ultrasonic probe 404 and transmission member 502 in its oscillated state, a state where the ultrasonic vibration is applied.

Considering the use of ultrasonic probe 404 in treatment procedures, longitudinal vibration would be the desirable ultrasonic vibration. On the contrary, transverse vibrations and torsional vibrations would be undesirable ultrasonic vibrations that may cause issues during the treatment procedures. Because the ultrasonic probe 404 is curved in the horizontal direction with an aim to improve the visibility during the treatment procedure, the axial unbalance of the ultrasonic probe 404 in the horizontal direction may create substantial transverse vibrations when the ultrasonic vibration is applied to the ultrasonic probe 404. In the case shown in FIG. 3B, the ultrasonic vibration has caused a strong transverse vibration at the antinodes 504, leading to problems such as deterioration of blood vessel sealing performance, heat generation, abnormal stress, and abnormal noise.

FIG. 4A illustrates the ultrasonic probe 404 viewed from the horizontal direction, the direction perpendicular to the vertical direction referred to in FIGS. 3A and 3B. FIG. 4A also illustrates the transmission member 502 extending from the ultrasonic probe 404, extending within the shaft 304, and connecting to the transducer 312. The ultrasonic probe 404 and transmission member 502 are in its stationary state, a state where neither the ultrasonic vibration nor the high frequency current is applied to the ultrasonic probe 404 and transmission member 502.

FIG. 4B also illustrates the ultrasonic probe 404 viewed from the horizontal direction. FIG. 4B illustrates an exaggerated representation of the ultrasonic probe 404 and the transmission member 502 in its oscillated state, a state where the ultrasonic vibration is applied. Because the ultrasonic probe 404 is not curved in the vertical direction, axial unbalance in the vertical direction is minimal compared to the axial unbalance due to the curved ultrasonic probe 404 curving in the horizontal direction. Thus, the undesired transverse vibrations that may occur at the antinode 504 at the time of application of ultrasonic vibration is weak compared to the transverse vibrations in the horizontal direction as disclosed in FIG. 3B. FIG. 5 also illustrates an exaggerated representation of the ultrasonic probe 404 and the transmission member 502 in its perspective view. FIG. 5 illustrates the ultrasonic probe 404 and transmission member 502 in its oscillated state, showing the occurrence of undesired transverse vibration created due to the curve of the ultrasonic probe 404.

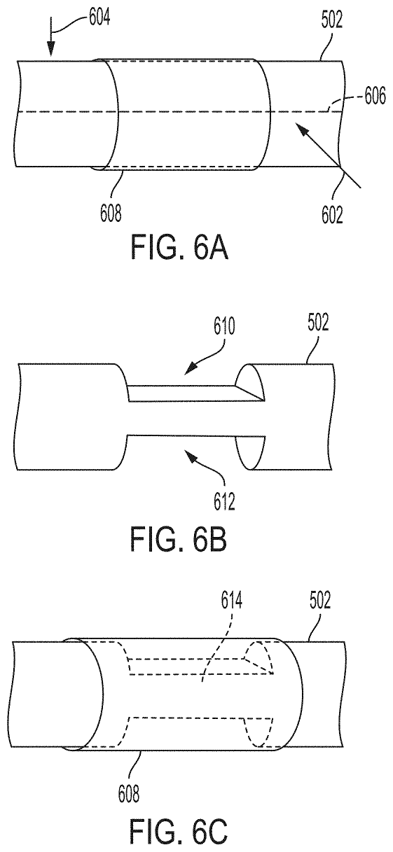

FIG. 6A illustrates the ultrasonic probe 404 viewed from the horizontal direction 602, the direction perpendicular to the vertical direction 604. The vertical view direction 604 is the same direction the transmission member 502 is viewed in FIGS. 3A and 3B, which is the direction the jaw 402 opens and closes. The transmission member 502, extending in the direction of center axis 606, is covered by a damping structure, such as an attenuation tube 608. The attenuation tube 608 comes in contact with the transmission member 502 and serves to attenuate the transverse vibrations caused by the ultrasonic vibration applied to the ultrasonic probe 404. The attenuation tube 608 may include a linear or helical slit for easing the attachment to the transmission member 502. Attenuation tube 608 may consist of a sleeve structure. In order to suppress the frictional heat caused by the ultrasonic vibration applied to the ultrasonic probe 404, it is preferred to place the attenuation tube 608 at the node or near the node of the longitudinal vibration. In order to attenuate the transverse vibration caused by the ultrasonic vibration applied to the ultrasonic probe 404, it is preferred to place the attenuation tube 608 at a location including at least one antinode of the transverse vibration. The attenuation tube 608 is made from polymer materials such as fluororesins, PTFE, FEP, and PFA with a thickness around 0.1 to 1.0 mm. The attenuation tube 608 may include a linear or helical slit for easing the attachment to the transmission member 502.

FIG. 6B also illustrates the transmission member 502 viewed from the side view 602. FIG. 6B discloses the upper notch 610 and lower notch 612 in the transmission member 502 that results from, for example, a portion of the transmission member 502 being removed, such as cut or scraped. The upper notch 610 and lower notch 612 serves to avoid the attenuation tube 608 to contact the transmission member 502 at the location of the notches. This configuration aims to concentrate the attenuation effort of the attenuation tube 608 to the undesired transverse vibration in the horizontal direction discussed in the description regarding FIG. 3B above, where the axial unbalance due to the horizontally curved portion of the ultrasonic probe 404 likely creates a strong transverse vibration compared to the transverse vibration in the vertical direction as discussed in the description regarding FIG. 3B.

FIG. 6C illustrates the notched transmission member 502 disclosed in FIG. 6B viewed from the side view 602, in combination with attenuation tube 608. Due to the upper notch 610 and lower notch 612, the transmission member 502 is only in contact with the attenuation tube 608 at the side surface 614 where there likely is a strong undesired transverse vibration in the horizontal direction. This configuration serves to attenuate the transverse vibration through the contacting of side surface 614 and attenuation tube 608, while maintaining the flexural rigidity of the ultrasonic probe 404 in the horizontal direction. Also, due to the lack of contact between the attenuation tube 608 and the surfaces of transmission member 502 at upper notch 610 and lower notch 612, the frictional heat due to the friction between the attenuation tube 608 and transmission member 502 caused by the longitudinal vibration would be significantly reduced.

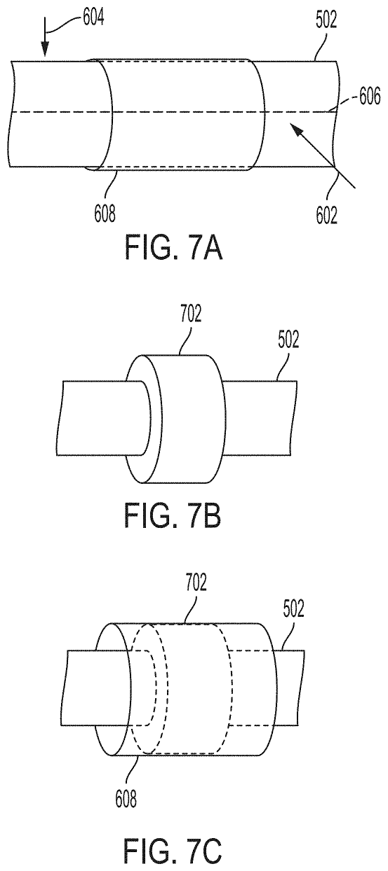

FIG. 7A illustrates the transmission member 502 viewed from the side view direction 602 perpendicular from the vertical view direction 604. The vertical view direction 604 is the same direction the transmission member 502 is viewed in FIGS. 3A and 3B, which is the view from the direction the jaw 402 is located.

The transmission member 502, extending in the direction of center axis 606, is covered by attenuation tube 608. The attenuation tube 608 comes in contact with transmission member 502 and serves to attenuate the due to transverse vibration caused by the ultrasonic vibrations applied to the transmission member 502.

FIG. 7B also illustrates the transmission member 502 viewed from the side view direction 602. FIG. 7B discloses the thickened portion 702 having larger diameter than the other portions of the ultrasonic probe 404. The thickened portion 702 is calculated to be placed at or near the antinode of the transverse vibration, viewed in terms of the axial direction, in order to increase the efficiency of the attenuation. The thickened portion 702 is also calculated to be placed at or near the node of the longitudinal vibration, viewed in terms of the axial direction, in order to minimize the frictional heat caused by the contact of the attenuation tube 608 and the thickened portion 702.

FIG. 7C illustrates the transmission member 502 having the thickened portion 702 disclosed in FIG. 7B viewed from the side view direction 602, in combination with attenuation tube 608. Due to the thickened portion 702, the transmission member 502 is only in contact with the attenuation tube 608 at the surface of the thickened portion 702. This configuration serves to attenuate the transverse vibration through the contacting of the thickened portion 702 with the attenuation tube 608, while avoiding contact of the between the attenuation tube 608 and surfaces of the portions other than the thickened portion 702 of the transmission member 502. Because the portions other than the thickened portion 702 would not be in contact with the attenuation tube 608, the frictional heat caused by the friction between the attenuation tube 608 and transmission member 502 due to the vertical transverse vibration would be significantly reduced.

Due to the thickened portion 702, the transmission member 502 is only in contact with the attenuation tube 608 at the thickened portion 702. This configuration serves to attenuate the transverse vibration through the contacting of thickened portion 702 and attenuation tube 608, while maintaining the flexural rigidity of the ultrasonic probe 404 by thickening the transmission member 502 at or near the antinode of the transverse vibration and thereby suppressing the transverse vibration. Also, due to the lack of contact between the attenuation tube 608 and the outer surface of transmission member 502, the frictional heat due to the friction between the attenuation tube 608 and longitudinal vibration of the transmission member 502 would be significantly reduced.

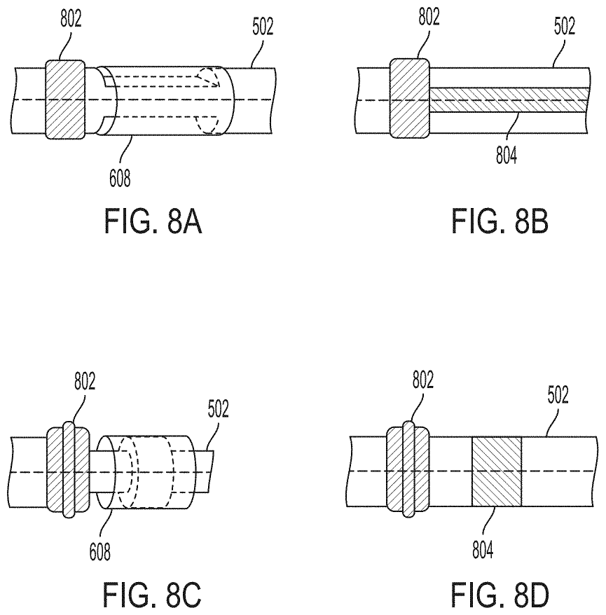

FIG. 8A illustrates the notched transmission member 502 disclosed in FIG. 6C in combination with attenuation tube 608 and rubber member 802. FIG. 8B illustrates the transmission member 502 without a notch and coated with coating material 804, made from materials such as PEEK resin, fluororesin, polyimide resin, ceramic, or rubber, having effects to attenuate transverse vibration on the side surface. If the coating material 804 is coated in the area equivalent to where the transmission member 502 and attenuation tube 608 makes contact in FIG. 8A and the coating material 804 has the equivalent attenuation efficiency as the attenuation tube 608 in combination with transmission member 502, the level of attenuation of the transverse vibration achieved would be equivalent in FIGS. 8A and 8B. Note that there would be less issues related to longitudinal vibrations for the structure disclosed in FIG. 8B due to lack of attenuation tube 608.

FIG. 8C illustrates the thickened transmission member 502 disclosed in FIG. 7C in combination with attenuation tube 608 and rubber member 802. FIG. 8D illustrates a transmission member 502 without a thickened portion and coated with coating material 804 having effects to attenuate transverse vibration coated on some portions of its circumference surface. If the coating material 804 is coated in the area equivalent to where the transmission member 502 and attenuation tube 608 makes contact in FIG. 8C and the coating material 804 has the equivalent attenuation efficiency as the attenuation tube 608 in combination with transmission member 502, the level of attenuation of the transverse vibration achieved would be equivalent in FIGS. 8C and 8D. Note that there would be less issues related to longitudinal vibrations for the structure disclosed in FIG. 8D due to lack of attenuation tube 608.

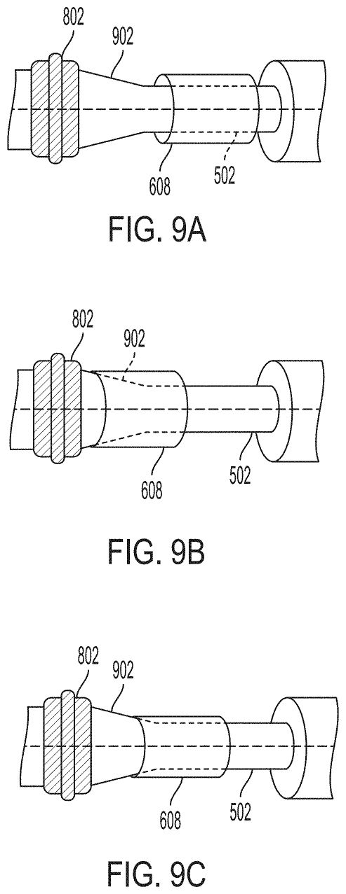

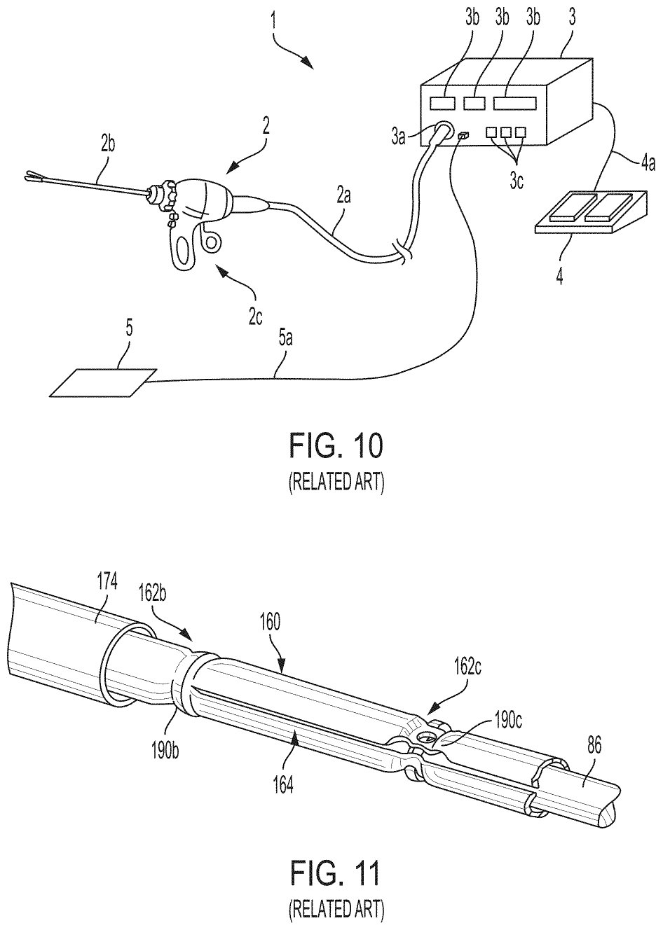

FIG. 9A illustrates the transmission member 502 including a tapered portion 902 in combination with attenuation tube 608 and rubber member 802. As illustrated in FIGS. 9B and 9C, upon oscillation of the ultrasonic vibration on the transmission member 502, the attenuation tube 608 moves to the distal end of the transmission member 502, away from the transducer 312, which is the source of the ultrasonic vibration. As illustrated in FIGS. 9B and 9C, the contact location of the attenuation tube 608 and transmission member 502 is determined by the inner diameter of the attenuation tube 608 and outer diameter of the tapered portion 902. Therefore, the contact location of the attenuation tube 608 and transmission member 502 may be set at the antinode of the transverse vibration in order to mitigate negative effects caused by the transverse vibration of the ultrasonic vibrations.

Although the present invention has been described in connection with preferred embodiments thereof, it will be appreciated by those skilled in the art that additions, deletions, modifications, and substitutions not specifically described may be made without department from the spirit and scope of the invention as defined in the appended claims.

What is claimed is:

1. A surgical treatment device, comprising:
a transducer generating ultrasonic vibrations;
a transmission rod including a treatment probe, wherein a proximal end of the transmission rod is operatively connected to the transducer for transmitting ultrasonic vibration generated by the transducer to the treatment probe located at the distal end; and
a damping feature for attenuating vibrations,
wherein the damping feature has an interior surface that circumscribes a first region of the transmission rod,
wherein the interior surface of the damping feature is in contact with a first portion of the outer surface of the first region of the transmission rod and the interior surface of the damping feature is in non-contact with a second portion of the outer surface of the first region of the transmission rod, and
wherein the first region includes at least one antinode of the transverse vibration.

2. The surgical treatment device according to claim 1, wherein the treatment probe includes a curved portion.

3. The surgical treatment device according to claim 1, wherein the first region includes a notch.

4. The surgical treatment device according to claim 1, wherein the first portion comprises two opposing outer surfaces of the first region.

5. The surgical treatment device according to claim 4, wherein the two opposing outer surfaces include the horizontal plane parallel to the direction of the curve of the curved portion.

6. The surgical treatment device according to claim 1, wherein the second portion comprises two opposing outer surfaces of the first region that are flat and parallel to each other.

7. The surgical treatment device according to claim 1, wherein the first portion does not include an antinode of the longitudinal vibration.

8. The surgical treatment device according to claim 1, wherein the damping feature is a sleeve.

9. The surgical treatment device according to claim 1, wherein the damping feature is a tube.

10. The surgical treatment device according to claim 1, wherein the damping feature is a coating material.

11. The surgical treatment device according to claim 1, wherein the damping feature includes a slit.

12. The surgical treatment device according to claim 1, wherein the transmission rod is configured as an electrode for treatment using high frequency currents.

13. A surgical treatment device, comprising:
a transducer generating ultrasonic vibration;
a transmission rod including a treatment probe, wherein a proximal end of the transmission rod is operatively connected to the transducer for transmitting ultrasonic vibration generated by the transducer to the treatment probe located at the distal end; and
a damping feature for attenuating vibrations,
wherein the damping feature has an interior surface that circumscribes an outer surface of a first region of the transmission rod,
wherein the interior surface of the damping feature is in contact with a first portion of the circumferential surface of the first region of the transmission rod and the interior surface of the damping feature is in non-contact with a second portion of the circumferential surface of the first region of the transmission rod, and
wherein the first region includes at least one antinode of the transverse vibration.

14. The surgical treatment device according to claim 13, wherein the first portion includes a node of a longitudinal vibration.

15. The surgical treatment device according to claim 13, wherein the first portion does not include an antinode of a longitudinal vibration.

16. The surgical treatment device according to claim 13, wherein the first portion does not include a node of the transverse vibration.

17. A transmission rod, comprising:
an elongate body configured for transmitting ultrasonic vibration from a proximal end to a distal end; and
a treatment probe formed at the distal end of the elongate body, wherein the treatment probe includes a treatment surface and a curved portion,
wherein the elongate body includes a notch covering the vertical vertex of the elongate body, and
wherein the notch includes an antinode of the transverse vibration.

18. The transmission rod according to claim 17, wherein a first portion of an outer surface of a first region of the transmission rod comprises two opposing outer surfaces.

19. The transmission rod according to claim 18, wherein the two opposing outer surfaces include the horizontal plane parallel to the direction of the curve of the curved portion.

20. The transmission rod according to claim 18, wherein a second portion of the outer surface of the first region of the transmission rod comprises two opposing outer surfaces that are flat and parallel to each other.
